(19) United States
(12) Patent Application Publication
(10) Pub. No.: US 2012/0196342 A1
(43) Pub. Date: Aug. 2, 2012

(54) Industrial Applications of A Novel Aldo/Keto Reductase Of Zymomonas Mobilis

(76) Inventors: Rachel Ruizhen Chen, Marietta, GA (US); Manoj Agrawal, Atlanta, GA (US)

(21) Appl. No.: 13/018,922

(22) Filed: Feb. 1, 2011

(51) Int. Cl. C12P 7/10; C12S 3/04; C12N 9/04; C12N 1/21

(52) U.S. Cl. 435/165; 435/252.3; 435/277; 435/190

(57) ABSTRACT

The present invention relates to methods of reducing the toxicity of lignocellulosic hydrolysates which comprise one or more inhibitors. One method reduces the amount of furfural inhibitor leading to a more effective process. Another method reduces the amount of xylitol produced during the fermentation of xylose present in lignocellulosic hydrolysates. Naturally occurring aldo/keto reductase enzymes, as well as, enzymes produced by recombinant cells or by selective adaptation may be employed.

### FIELD OF THE INVENTION

The instant invention pertains to, for example, a novel enzyme important to xylose fermentation in Zymomonas mobilis (Z. mobilis) and which can be used to detoxify lignocellulosic hydrolysates by converting microbial growth inhibitors such as furfural to a non-toxic alcohol.

### BACKGROUND AND SUMMARY OF THE INVENTION

Sugars obtained from hydrolysis of lignocellulosic biomass are used in many processes. For example, the process of ethanol production using biomass as a feedstock is well known (http://www.vermontbiofuels.org/biofuels/ethanol.shtml). In this process, both hexoses and pentoses are fermented to ethanol by a microorganism. Currently, yeast (Saccharomyces cerevisiae) is often used in this fermentation process, see, Almeida, J. R. M., et al., J. of chem. tech. and biotech., 2007, 82(4): pp. 340-349. Z. mobilis has also been used in this fermentation process and through metabolic engineering a Z. mobilis strain has been developed to ferment xylose, (see, Zhang, M., Engineering Zymomonas mobilis for efficient ethanol production from lignocellulosic feedstocks, ACS national meeting, 2003 and U.S. Pat. No. 7,223,575, which is incorporated herein by reference to the extent that it is not inconsistent) and as well as arabinose, see, Mohagheghi, A., et al., Applied biochemistry and biotechnology, 2002, 98-100: pp. 885-898.

Unfortunately, Z. mobilis and other fermenting microorganisms often suffer from a toxicity issue, i.e., they are very sensitive, to various chemicals including ethanol, aliphatic acids, such as acetic acid, formic acid; furan derivatives, such as 2-furaldehyde, 2-furoic acid; and phenolic compounds, such as vanillin and hydroxybenzoic acid, found in the biomass, see, Lawford, H. G., et al., Applied biochemistry and biotechnology, 1993, 39/40: pp. 687-699. Thus, before using Z. mobilis in industry, the inhibition problem has to be addressed.

Recently, Chen et al. successfully modified Z. mobilis to develop inhibitor tolerance and/or enhanced pentose, e.g., xylose, consumption rates using selective pressure methods. See, for example, PCT Publication No. WO/2009/132201 which is hereby incorporated by reference to the extent that it is not inconsistent. However, one of the existing drawbacks of xylose fermentation is the formation of side product xylitol by xylose reductase (XR) activity in Z. mobilis ZM4. Identity of XR in Z. mobilis has remained elusive since 1992 when the first reported unsuccessful attempt at constructing xylose fermenting strain was made by Feldmann, S. D., H. Sahm, et al., “Pentose metabolism in Zymomonas mobilis wild-type and recombinant strains,” Applied Microbiology and Biotechnology 38(3): 354-361. This ignorance has been pointed out again recently by researchers. See, for example, Viitanen, McCutchen, et al., Xylitol synthesis mutant of xylose-utilizing Zymomonas for ethanol production, PCT Publication No. WO/2008/133,638 and Zhang, Chen, et al., “Reduction of xylose to xylitol catalyzed by glucose-fructose oxidoreductase from Zymomonas mobilis,” Fems Microbiology Letters 293(2): pp. 214-219. The identification of XR in Z. mobilis is important since it is believed that the enzyme(s) results in the production of xylitol which inhibits xylose fermentation by engineered Z. mobilis. Thus, it would be desirable to discover a way of reducing the xylitol formation during conversion of lignocellulosic derived xylose by Z. mobilis to commercial products such as ethanol.

Furfural and HMF are produced during hydrolysis of lignocellulosic biomass and like xylitol may be potent inhibitors of microbial growth. For example, the presence of 7.3 mM furfural or 9.5 mM HMF can reduce the growth rate of Z. mobilis by 25% while at a concentration of 52 mM furfural or 63 mM HMF, the growth is completely inhibited according to Franden, Pienkos, et al., “Development of a high-throughput method to evaluate the impact of inhibitory compounds from lignocellulosic hydrolysates on the growth of Zymomonas mobilis,” Journal of Biotechnology 144(4): 259-267. Various ethanol fermentation microbes are able to partially reduce these aldehydes to corresponding alcohols and thus partially reduce the toxicity associated with furfural and HMF. Thus, it would be desirable to discover these microbial enzymes and employ them for detoxifying lignocellulosic hydrolysates. There has been at least one attempt to characterize bacterial furfural reductase by Gutierrez, T., L. O. Ingram, et al., “Purification and characterization of a furfural reductase (FFR) from Escherichia coli strain LYO1—An enzyme important in the detoxification of furfural during ethanol production,” Journal of Biotechnology 121(2): pp. 154-164.

Advantageously, the instant invention relates in one embodiment to a way of treating a lignocellulosic hydrolysate comprising one or more inhibitors. The method comprises: contacting a lignocellulosic hydrolysate comprising one or more inhibitors with an aldo/keto reductase. It is contacted under conditions such that the total amount of inhibitors such as furfural is reduced. The aldo/keto reductase may be employed neat, in a solution, or generated in situ from one or more cells, e.g., recombinant cells, capable of producing said aldo/keto reductase.

Advantageously, the instant invention relates in another embodiment to a method for reducing xylitol production during xylose fermentation by recombinant Z. mobilis. By a single mutation of ZM00976 to yield mZM00976, the xylitol production can be decreased. Thus, a method comprising fermentation of xylose by recombinant Z. mobilis containing mZM00976, instead of ZMO0976, results in a greater ethanol yield and higher cell growth as compared to prior art methods since the formation of inhibitor xylitol is reduced. The enzyme, ZMO0976, in this patent application appears to be novel in its activity towards xylose. Thus, the method for fermenting xylose-containing lignocellulosic hydrolysate comprises fermenting a xylose-containing lignocellulosic hydrolysate in the presence of (1) recombinant Z. mobilis which synthesizes mZMO0976 or a suitable derivative thereof in the substantial absence of ZMO0976; or (2) recombinant Z. mobilis rendered incapable of synthesizing ZMO0976 or a suitable derivative thereof; or (3) a combination of (1) and (2).

Advantageously, the instant invention relates in another embodiment to an aldo/keto reductase from a recombinant cell. The aldo/keto reductase comprises the amino acid sequence ZM00976 and has one or more of the following characteristics: (1) a xylose reductase activity of 3400±200 mU/mg protein; (2) a furfural reductase activity of 5470±60 mU/mg protein; (3) a benzaldehyde reductase activity of 4030±250 mU/mg protein; (4) an acetaldehyde reductase activity of 2500±400 mU/mg protein; and (5) ability to reduce HMF present in lignocellulosic biomass.

In another embodiment, the invention pertains to a recombinant cell capable of producing an aldo/keto reductase useful for treating or detoxifying a lignocellulosic hydrolysate. The recombinant cell comprises a nucleic acid molecule capable of encoding the amino acid sequence ZM00976 or a suitable derivative.

### BRIEF DESCRIPTION OF THE FIGURES

FIG. 1 illustrates a plasmid map for pQEZM976 or pQEmZM976*.

FIG. 2 illustrates a comparison of xylitol produced by xylose-fermenting adapted strain A3 and non-xylose fermenting control strain ZM4/pSTVZM27 of Z. mobilis just after the consumption of all the 5% glucose in a 5% glucose-5% xylose batch fermentation.

FIG. 3 illustrates SDS-PAGE for cell-free extracts (CFEs) and immobilized metal-ion affinity chromatography (IMAC)-purified CFEs of UT5600/pQE80L, UT5600/pQEZM976 and UT5600/pQEmZM976*. From left: Lane (L) 1 & L10: Protein ladder, L2: CFE of UT5600/pQE80L, L4: CFE UT5600/pQEZM976 (Duplicate 1), L5: Purified protein for CFE UT5600/pQEZM976 (Duplicate 1), L6: CFE UT5600/pQEmZM976*, L7: Purified protein for UT5600/pQE mZM976*, L8: CFE UT5600/pQEZM976 (Duplicate 2), L9: Purified protein for CFE UT5600/pQEZM976 (Duplicate 2). 15 μg of CFE and 0.75 μg of IMAC-purified CFE were loaded into each well.

FIGS. 4a and 4b are a 341 amino acid protein described as aldo/keto reductase ZM00976 and mZM00976, respectively. The site of mutation (292^{nd} amino acid residue) is shown by a bold, capitalized, underlined letter.

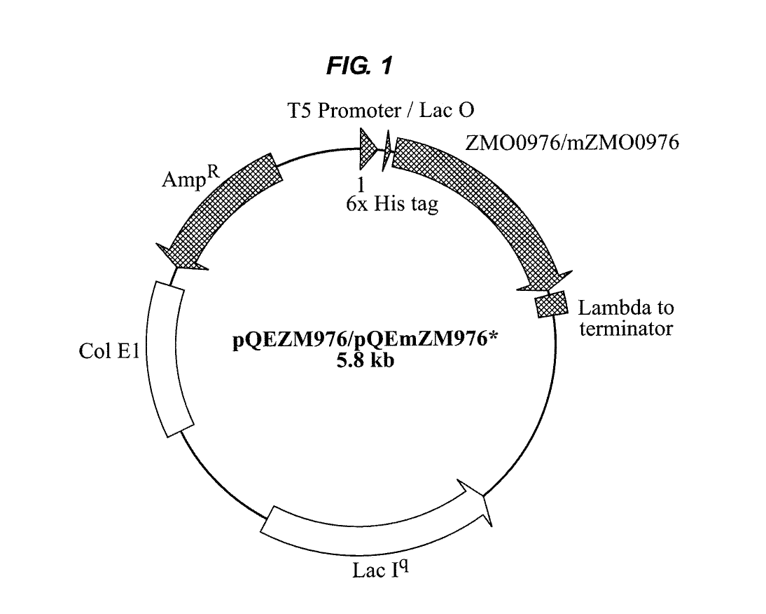

### DETAILED DESCRIPTION OF THE INVENTION

### Definitions

The term “fermentable sugar” refers to oligosaccharides and monosaccharides that can be used as a carbon source by, for example, Z. mobilis in a fermentation process.

The term “lignocellulosic” refers to a composition comprising both lignin and cellulose. Lignocellulosic material may also comprise hemicellulose.

The term “lignocellulosic hydrolysate” refers to lignocellulosic material that has been subjected to hydrolysis. The hydrolysis may be by any convenient method to depolymerize the material.

The term “biomass” includes untreated biomass or treated biomass, e.g., biomass that has been treated in some manner prior to saccharification. Generally, biomass includes any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

The term “suitable fermentation conditions” refers to conditions that support the production of ethanol using, for example, a Z. mobilis strain. Such conditions may include suitable pH, nutrients and other medium components, temperature, atmosphere, and other environmental factors.

The term “suitable derivative” as used in “ZM00976 or a suitable derivative thereof” and “mZM00976 or a suitable derivative thereof” refers to amino acid sequences that have substantially the same activity but may have one or more amino acids that differ. That is, amino acids may differ so long as the desired activity with regard to the substrates, e.g. furfural, is not degraded significantly.

### Processes Generally

Any lignocellulosic hydrolysate may benefit from the embodiments of the instant invention that reduce the amount of inhibitors and/or lessen the toxicity so that the sugars in the lignocellulosic hydrolysate are more useful in subsequent processing like fermentation. Cellulose is the most common form of carbon in biomass, accounting for 40%-60% by weight of the biomass, depending on the biomass source. It is a complex sugar polymer, or polysaccharide, made from the six-carbon sugar, glucose. Hemicellulose is also a major source of carbon in biomass, at levels of between 20% and 40% by weight. It is a complex polysaccharide made from a variety of five- and six-carbon sugars.

The complex polysaccharides in the biomass may be converted by, for example, hydrolysis to sugars by treatment with steam, acid, alkali, cellulases or combinations thereof. Using the embodiments of the instant invention the sugars may be rendered less toxic so that they can then be used as desired, for example, converted to ethanol by fermentation. Such fermentation may include using, for example, a strain of Z. mobilis or other microbe which occurs naturally, is obtained by selective adaptation, or is made via recombinant DNA technology. In certain embodiments, the sugars comprise glucose, fructose, sucrose, xylose, arabinose, mannose or a mixture thereof.

### General Process to Reduce Amount of Furfural Inhibitor in Bioalcohol Production

In one embodiment the invention relates to a method for treating a lignocellulosic hydrolysate comprising one or more inhibitors. The method comprises contacting a lignocellulosic hydrolysate comprising one or more inhibitors with an aldo/keto reductase under conditions such that the total amount of inhibitors is reduced.

The contacting of lignocellulosic hydrolysate and aldo/keto reductase may be employed under any conditions in which the total amount of furfural present is reduced. Generally, the aldo/keto reductase is employed neat, employed in a solution, or generated in situ from one or more cells capable of producing said aldo/keto reductase. That is so long as a suitable aldo/keto reductase is employed, it may originate from any source. In one embodiment, the aldo/keto reductase may be generated in situ from one or more recombinant cells made capable of producing said aldo/keto reductase.

The specific aldo/keto reductase is not particularly critical so long as it is capable of reducing the total amount of inhibitors present. Thus, in some embodiments the aldo/keto reductase reduces the amount of one, or two, or three or more inhibitors selected from the group consisting of furfural, benzaldehyde, acetaldehyde, and HMF.

The manner of reduction is not particularly critical so long as the amount of total inhibitors is reduced. Therefore, in one embodiment the reduction occurs through modifying the furfural to a compound which does not subsequently cause significant interference with fermentation. Thus, in one embodiment the aldo/keto reductase reduces the amount of furfural by converting furfural to 2-furanmethanol. In some embodiments, the aldo/keto reductase is also advantageously capable of reducing the amount of one, two, or all three of the following compounds: benzaldehyde, acetaldehyde, and HMF.

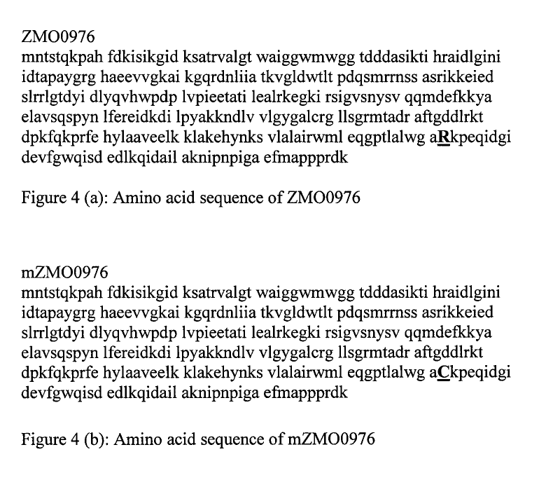

One particularly useful aldo/keto reductase comprises an amino acid sequence of ZM00976 shown in FIG. 4a or a suitable derivative thereof. Such aldo/keto reductase may result from or be derived from naturally occurring Z. mobilis. Alternatively, such aldo/keto reductase may result from or be derived from a recombinant microbe that has been genetically modified to produce said amino acid sequence. That is, useful recombinant cells include those that comprise a nucleic acid molecule capable of encoding the amino acid sequence ZM00976. Useful aldo/keto reductase in the present invention from natural or recombinant cells may have one or more, or two or more, or even three or more of the following characteristics: (1) a xylose reductase activity of 3400±200 mU/mg protein; (2) a furfural reductase activity of 5470±60 mU/mg protein; (3) a benzaldehyde reductase activity of 4030±250 mU/mg protein; (4) an acetaldehyde reductase activity of 2500±400 mU/mg protein; and (5) ability to reduce HMF present in lignocellulosic biomass. In addition, in some instances the aldo/keto reductase may advantageously be capable of reducing other aldehydes or even oxidizing alcohol to aldehyde.

If necessary or desired, one or more cofactors may be employed with the aldo/keto reductase. The specific type and amount of cofactor may vary depending upon, for example, the specific aldo/keto reductase and desired results. A particularly effective cofactor for use with the aforementioned ZM00976 may include NADPH which can then be recycled for subsequent use in any convenient manner. Once one or more inhibitors have been reduced the lignocellulosic hydrolysate may be subjected to subsequent processing such as fermentation.

### General Process to Ferment Xylose-Containing Lignocellulosic Hydrolysate

In another embodiment, the instant invention relates to a method for reducing xylitol formation during fermentation of xylose present in lignocellulosic hydrolysate by Z. mobilis. The method comprises using recombinant xylose-fermenting Z. mobilis for a fermentation process that possesses (1) mZMO0976 (amino acid sequence shown in FIG. 4b) or a suitable derivative thereof; or (2) ZMO0976 gene knocked out. The recombinant xylose-fermenting Z. mobilis containing ZMO0976 results in higher xylitol production, which inhibits the xylose fermentation and cell growth.

The instant process typically assists with toxicity by reducing the amount of xylitol which may serve as an inhibitor. For example, a reduced amount of xylitol is often produced in the instant process involving mZMO0976 or a derivative as compared to ZMO0976 or a derivative. In one embodiment, Z. mobilis cells which synthesize the amino acid sequence mZMO0976 may be produced according to the selective pressure methods described in, for example, PCT Publication No. WO/2009/132201 incorporated herein by reference. Alternatively, recombinant cells that synthesize the amino acid sequence mZMO0976 or a derivative thereof may be produced using methods for recombinant DNA technology that are known in the art. Additionally, the gene responsible for ZMO0976 could be knocked out. Exemplary methods are described by Baumler et al. in Applied biochemistry.

Thus, a method for fermenting xylose-containing lignocellulosic hydrolysate may be employed. The method comprises fermenting a xylose-containing lignocellulosic hydrolysate in the presence of (1) recombinant Z. mobilis which synthesizes mZMO0976 or a suitable derivative thereof in the substantial absence of ZMO0976; or (2) recombinant Z. mobilis rendered incapable of synthesizing ZMO0976 or a suitable derivative thereof; or (3) a combination of (1) and (2). As described previously, a recombinant Z. mobilis rendered incapable of synthesizing ZMO0976 may be employed by knocking out the appropriate gene using any convenient method. Similarly, recombinant cells capable of producing an aldo/keto reductase useful for treating a lignocellulosic hydrolysate may be made in any suitable manner. Such recombinant cells typically comprises a nucleic acid molecule capable of encoding the amino acid sequence ZM00976 or a suitable derivative thereof.

### Fermentation or Other Subsequent Processing

After inhibitors have been reduced using one or more of the aforementioned techniques, the sugars may be usefully employed in many processes. One such process is fermentation. Suitable fermentation conditions are known in the art. Substrate concentrations of up to about 25% (based on glucose), and under some conditions even higher, may be used. Unlike other ethanol producing microorganisms, no oxygen is needed at any stage for Z. mobilis survival. Also, unlike yeast, oxygen does not drastically reduce ethanol productivity or greatly increase cell growth. Agitation is not necessary but may enhance availability of substrate and diffusion of ethanol. Accordingly, the range of fermentation conditions may be quite broad. Likewise, any of the many known types of apparatus may be used for the production of ethanol by the process.

Fermentation can be carried out in a bioreactor, such as a chemostat, tower fermenter or immobilized-cell bioreactor. In certain embodiments, fermentation is carried out in a continuous-flow stirred tank reactor. Mixing can be supplied by an impeller, agitator or other suitable means and should be sufficiently vigorous that the vessel contents are of substantially uniform composition, but not so vigorous that the microorganism is disrupted or metabolism is inhibited.

The fermentation process may be carried out as a batch process or parts or all of the entire process may be performed continuously. To retain the microorganisms in the fermenter, one may separate solid particles from the fluids. This may be performed by centrifugation, flocculation, sedimentation, filtration, etc. Alternatively, the microorganisms may be immobilized for retention in the fermenter or to provide easier separation.

Microbes such as Z. mobilis strains may be used as a biologically pure culture or may be used with other ethanol producing microorganisms in mixed culture. In certain embodiments, preexisting deleterious microorganisms in the substrate are eliminated or disabled before adding strains to the substrate. In certain embodiment, enzyme(s) are added to the fermenter to aid in the degradation of substrates or to enhance ethanol production. For example, cellulase may be added to degrade cellulose to glucose simultaneously with the fermentation of glucose to ethanol by microorganisms in the same fermenter. Likewise, a hemicellulase may be added to degrade hemicellulose.

In certain embodiment, the process for ethanol production is optimized for maximum ethanol production by various techniques known to one of skill in the art, including, but not limited, to removal of one or more inhibitors, for example acetic acid, formic acid, 2-furaldehyde, 2-furoic acid, vanillin and hydroxybenzoic acid, from the pretreated biomass, finding more optimal fermentation conditions. Exemplary techniques for removal of acetic acid from the pretreated biomass include, but are not limited to, use of ion-exchange resins and ion exchange membranes. As one of skill will appreciate, the fermentation conditions may be improved by taking into consideration both biomass and sugar utilization when selecting the conditions as both may be factors.

After fermentation, the ethanol may be separated from the fermentation broth by any of the many conventional techniques known to separate ethanol from aqueous solutions. These methods include evaporation, distillation, solvent extraction and membrane separation. Particles of substrate or microorganisms may be removed before ethanol separation to enhance separation efficiency.

Once the fermentation is complete, microorganisms and unfermented substrate may be either recycled or removed in whole or in part. If removed, the microorganisms may be killed, dried or otherwise treated. This mixture may then be used as animal feed, fertilizer, burnt as fuel or discarded.

Unless specifically defined otherwise, all technical or scientific terms used herein have the same meaning as commonly understood by one of ordinary skill in the art to which this invention belongs. Although any methods and materials similar or equivalent to those described herein can be used in the practice or testing of the present invention, the preferred methods and materials are better illustrated by the use of the following non-limiting examples, which are offered by way of illustration and not by way of limitation.

### EXAMPLES

The following examples are presented to further illustrate and explain the claimed subject matter and should not be taken as limiting in any regard.

### Organisms and Media

Z. mobilis ZM4 was grown in rich media (RM) containing 1% yeast extract, 0.2% KH₂PO₄ and different amounts of glucose or xylose (as mentioned) as carbon source. Antibiotic selection marker, chloramphenicol 100 μg/ml was added for culturing engineered strains of ZM4. Escherichia coli (E. coli) K-12 substr. UT5600 were grown in Luria-Bertani (LB) media. Ampicillin 100 μg/ml was added to the media as needed.

### Culture Conditions

E. coli cells were grown at 37° C. in culture tubes or shake flasks at 250 rpm. E. coli cells were induced with 0.5 mM IPTG (Isopropyl β-D-1-thiogalactopyranoside) at an optical density (OD) of 0.4-0.6. Cells were grown for 4 hours at reduced temperature of 30° C. and then harvested for enzymatic assay.

Z. mobilis was grown at 30° C. Pre-seed culture (PSC) and seed culture (SC) of Z. mobilis were cultivated in 15-ml centrifuge tubes and 100-ml Pyrex screw-cap bottles respectively filled to 60% volume and shaken at 250 rpm. PSC was prepared by inoculating a single colony from agar plate (containing 5% xylose) into the liquid RM containing 2.5% glucose and 2.5% xylose. PSC was grown till the stationary phase. SC was prepared by inoculating it to an OD of 0.1 using the stationary phase PSC. SC contained 5% glucose and 5% xylose (5% G-5% X). Appropriate amounts of cells were harvested from SC at exponential phase, resuspended in fresh media, and were used to inoculate the main fermentation of RM containing 5% G-5% X carried out in fermenter to get a starting OD of 0.1. The fermenter setup is as generally described in Agrawal et al., “Adaptation yields a highly efficient xylose-fermenting Zymomonas mobilis strain,” Biotechnology and Bioengineering, n/a. doi: 10.1002/bit.23021).

The fermenter (Infors HT Multifors, Bottmingen, Switzerland) was stirred at 300 rpm, with minimum pH held at 5.75 by automatic addition of 1.7M KOH. N₂ purging was not done. To maintain anaerobic conditions, the exhaust tube was immersed in a water column to prevent atmospheric oxygen from diffusing into the fermenter vessels. Dissolved oxygen was monitored and was found to remain close to 0% throughout the fermentation. Three replicates were done for each experiment starting from single independent colonies on agar plate.

### Cloning of ZMO0976 from Z. mobilis into E. coli

The gene ZMO0976 was cloned from wild-type Z. mobilis ZM4 and its mutated form, mZMO0976 from A3 (xylose adapted strain of rationally engineered Z. mobilis ZM4/pZMETX for xylose metabolism) wherein A3 is as described in PCT Publication No. WO/2009/132201. The cloned gene was ligated at restriction sites KpnI and HindIII into the commercially available high copy number plasmid pQE80L (QIAGEN). The ligated vectors were named pQEZM976 (containing ZMO0976) and pQEmZM976* (containing mZMO0976). Both these vectors contained in-frame N-terminal histidine (His)₆-tag before the start codon of the genes and the genes are under the control of IPTG-inducible T5 promoter. These vectors were then transformed into E. coli UT5600 to construct UT5600/pQEZM976 and UT5600/pQEmZM976*, respectively. pQE80L was transformed into E. coli UT5600 to construct the control strain UT5600/pQE80L.

### Protein Purification & Enzymatic Assays

E. coli cells were prepared for enzymatic assays using the procedure described in, for example, Akinterinwa, and Cirino, “Heterologous expression of D-xylulokinase from Pichia stipitis enables high levels of xylitol production by engineered Escherichia coli growing on xylose,” Metabolic Engineering 11(1): pp. 48-55. Briefly, cells were harvested by centrifugation at 5,000 g at 4° C. for 30 min. Cells were washed once with extraction buffer [10 mM Tris-HCl pH 7.5, 5 mM MgCl₂, 1 mM dithothreitol (DTT) and 1 mM phenylmethylsulfonyl fluoride (PMSF) (Sigma-Aldrich, St. Louis, Mo.)]. Washed cell pellets were stored at −20° C. until use. Cell pellets were resuspended to an OD of 50 in equilibration buffer (50 mM Na₂HPO₄, 300 mM NaCl and 10 mM imidazole at pH 8 (Sigma-Aldrich, St. Louis, Mo.)) supplemented with 1 mM DTT and 1 mM PMSF, and sonicated (8 cycles of 10 s with 30 s cooling period). Cell debris was discarded after centrifugation at 16,000 g for 20 min at 4° C. Supernatant was used as cell-free extract (CFE) for enzymatic assays. Bradford Assay was used for estimation of protein concentration in the extract. His-tagged protein in the cell-free extract was purified at 4° C. using HIS-Select® HF Nickel Affinity Gel (Sigma-Aldrich, St. Louis, Mo.), which employs immobilized metal-ion affinity chromatography (IMAC). Manufacturer's protocol for large scale purification was followed. Briefly, 10 ml of cell-free extract was added to 1 ml of affinity gel in equilibration buffer. After overnight incubation, the gel was washed five times with equilibration buffer. The bound his-tagged protein was then eluted using 2 ml of elution buffer (50 mM Na₂HPO₄, 300 mM NaCl and 250 mM imidazole at pH 8). Imidazole was removed by overnight dialysis at 4° C. using extraction buffer as the dialysis solution.

Cell-free extract for Z. mobilis was prepared as above except that extraction buffer was used for sonication and after which, cell debris were spun down at higher centrifugal force (53,300 g). All enzymatic assay reactions were carried out in a final volume of 200 μl on a microplate. NAD(P)H (Sigma-Aldrich, St. Louis, Mo.) absorbance was measured at 340 nm using Spectramax M5 Plus spectrophotometer (Molecular devices, Sunnyvale, Calif.). Xylose reductase was assayed in McIlvaine buffer at pH 7.2 (prepared by adding 16.5 ml of 0.2 M Na₂HPO₄ to 3.5 ml of 0.1 M citric acid (Sigma-Aldrich, St. Louis, Mo.)) containing 0.35 mM NADPH or NADH (as indicated) and an appropriate volume of sample (cell-free extract or purified protein) according to Viikari, L. and M. Korhola (1986), “Fructose metabolism in Zymomonas mobilis,” Applied Microbiology and Biotechnology 24(6): 471-476.

Xylose concentration in the assay mixture is indicated in the text. For measuring the catalytic activity of xylose reductase towards other substrates, similar assay conditions were used. Kₘ and Vₘₐₓ were determined by varying the substrate concentrations in the assay mixture and fitting the data to Lineweaver-Burk equation: 1/v=(Kₘ/Vₘₐₓ) [S]+1/Vₘₐₓ. Xylitol concentration was determined enzymatically. The reaction was carried out with a total volume of 200 μl in 42 mM Tris-HCl buffer at pH 8.5, 5 mM NAD, 10 mM MgSO₄ (Sigma-Aldrich, St. Louis, Mo.), 4 U/ml sorbitol dehydrogenase (Roche Diagnostics, Indianapolis, Ind.) and 10 μl of sample. Xylitol concentration was estimated based on the amount of NADH produced by xylitol containing samples compared to that produced by xylitol standards. SDS-PAGE and Coommassie blue staining were used to confirm the expression of ZMO0976 and mZMO0976 proteins. 15 μg of cell-free extract protein and 1 μg of purified protein were loaded on a 12% Tris-HCl gel (Bio-Rad, Hercules, Calif.). Cell growth was determined by measuring optical density (OD) at 600 nm using spectrophotometer (Beckman Coulter DU 530, Brea, Calif.).

### Results

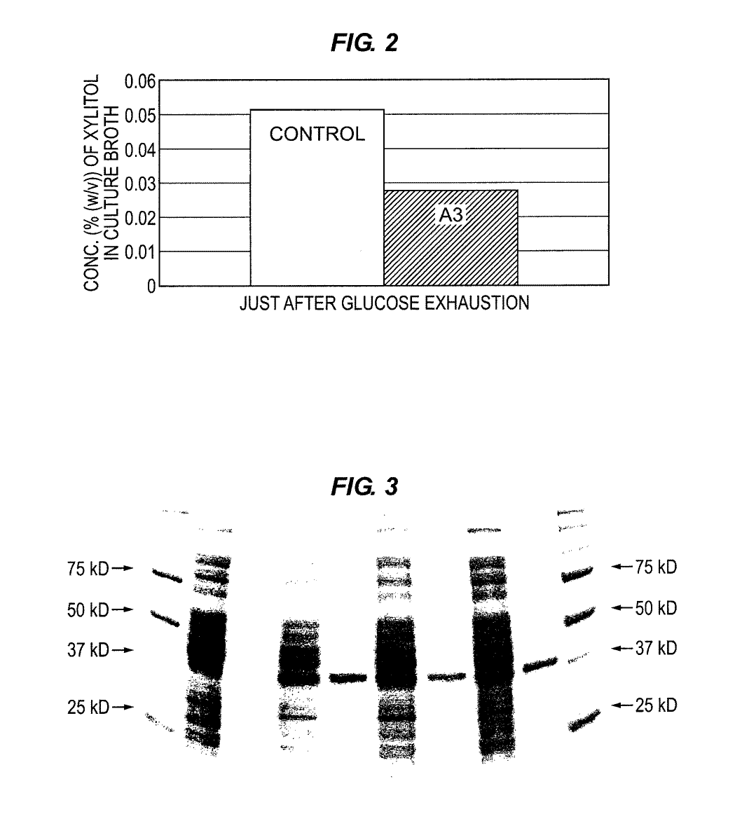

As shown in FIG. 2, adapted strain A3 produced nearly two-fold lower xylitol than the control strain ZM4/pSTVZM27 on fermentation of 5% glucose-5% xylose mixture just after the exhaustion of glucose from the media. Since A3 has been transformed with a plasmid harboring xylose metabolizing genes and subsequently adapted on xylose, it can grow on xylose whereas control strain, just like the wild-type Z. mobilis, can only grow on glucose.

Enzymatic assay for xylose reductase (XR) in cell-free extracts gave an activity of 22 mU/mg protein for the control strain, comparable to a previous report by Feldmann et al. (1992) for Z. mobilis strain CP4 (“Pentose metabolism in Zymomonas mobilis wild-type and recombinant strains,” Applied Microbiology and Biotechnology 38: pp. 354-361), whereas no activity could be detected in the cell extract of A3. Thus, the absence of XR activity in the adapted strain A3 suggested that the xylose reductase(s) may have been mutated. Using the NCBI's sequence analysis program tblastn, the amino acid sequences of characterized XRs from several fungi (including Aspergillus niger according to Prathumpai, M., J. Visser, et al. (2005), “Metabolic Control Analysis of Aspergillus niger L-Arabinose Catabolism,” Biotechnology Progress 21(6); Candida guilliermondii according to Handumrongkul, C., D. Ma, et al. (1998), “Cloning and expression of Candida guilliermondii xylose reductase gene (xyl1) in Pichia pastoris,” Applied Microbiology and Biotechnology 49(4): pp. 399-404; and Kluyveromyces lactis according to Billard, P., S. Ménart, et al. (1995), “Isolation and characterization of the gene encoding xylose reductase from Kluyveromyces lactis,” Gene 162(1): p. 93-97) were aligned against the Z. mobilis ZM4 genome. One gene, ZMO0976, annotated as aldose reductase, showed significant sequence identity (22%-31%) to these fungal XRs. Subsequent cloning and sequencing of the ZMO0976 gene from both the wild-type strain ZM4 and adapted strain A3 were carried out. A comparison of sequence showed a single base pair mutation, C874T, for the gene cloned from A3, which resulted in a single mutation from arginine to cysteine.

### Expression and Purification of ZMO0976 and mZMO0976

The gene ZMO0976 was cloned from ZM4 and its mutated form, mZMO0976 from A3 and expressed in E. coli UT5600 to construct UT5600/pQEZM976 and UT5600/pQEmZM976*, respectively. Thus, UT5600/pQEZM976 has ZMO0976 (unmutated form) under the control of IPTG-inducible T5 promoter. UT5600/pQEmZM976* instead has mZMO0976.

Cell-free extracts of both strains, UT5600/pQEZM976 and UT5600/pQEmZM976*, along with the control strain UT5600/pQE80L, were tested for NADPH-dependent xylose reductase activity. As shown in Table 1, the XR activity in the CFE for cells expressing ZMO0976 was high, 460 mU/mg protein, whereas cells expressing mZMO0976 exhibited a much diminished activity of 8 mU/mg protein. As expected, no activity was detected from cell extract of the control, UT5600/pQE80L. The native and mutated forms of the recombinant enzyme were purified based on the N-terminal His-tag. The purified proteins ZMO0976 and mZMO0976 had an expected molecular weight of ˜38 kDa as observed on SDS-PAGE (FIG. 3). XR activities of the purified proteins are 3400 and 140 mU/mg for ZMO0976 and the mZMO0976, respectively. Thus, the single mutation in ZMO0976 caused a significant reduction of XR activity.

TABLE 1
Xylose reductase activity of purified
protein and cell-free extract (CFE)

| Samples | Activity (mU/mg protein) |
|---|---|
| UT5600 expressing ZMO0976 (CFE) | 460 ± 50 |
| UT5600 expressing mZMO0976 (CFE) | 8 ± 2 |
| Purified recombinant ZMO0976 | 3400 ± 200 |
| Purified recombinant mZMO0976 | 140 ± 50 |

### Activity of ZMO0976 Towards Other Substrates and Co-Factors

With NADPH as cofactor, besides xylose, ZMO0976 showed activity towards benzaldehyde, furfural, acetaldehyde and 5-hydroxymethylfurfural (HMF), but negligible activity towards glucose and fructose. See Table 2 below.

TABLE 2
XR activities of recombinant ZMO0976 with different substrates

| | | Relative |
|---|---|---|
| Substrate | Conc. (mM) | activity |
| Benzaldehyde | 2 | 1 |
| Acetaldehyde | 260 | 0.62 |
| Furfural | 5.2 | 0.81 |
| HMF | 10 | 0.11 |
| Xylose | 260 | 0.83 |
| Glucose | 260 | 0 |
| Fructose | 260 | 0 |

To evaluate the single amino acid mutation on the enzyme activity toward substrates other than xylose, the mutated form of ZMO0976 was also purified and tested against furfural, benzaldehyde and acetaldehyde. As shown in Table 3, mZMO0976 had only a fraction of the activities of the wild type, indicating, as with xylose, a drastic impact of the single mutation on the activity. This effect was apparently independent of the substrates.

TABLE 3
Specific XR activities ZMO0976 and mZMO0976

| | Furfural | Benzaldehyde | Acetaldehyde |
|---|---|---|---|
| | (10 mM) | (2 mM) | (520 mM) |
| ZMO0976 | 5470 ± 60 | 4030 ± 250 | 2500 ± 400 |
| mZMO0976 | 180 ± 30 | 40 ± 10 | 170 ± 30 |

For three representative substrates, xylose, benzaldehyde, and furfural, respective Michaelis-Menten kinetics parameters were determined. For xylose, Kₘ and Vₘₐₓ were 258 mM and 6.9 U/mg protein, respectively. The high Kₘ value is consistent with the early observation that detectable activity requires a high concentration of xylose. The Kₘ for benzaldehyde was 1.77 mM, about 150 fold lower than xylose. The Kₘ for furfural, 4.15 mM, was also much lower than that of xylose (Table 4). These data indicate that ZMO0976 is more active on benzaldehyde and furfural than xylose.

TABLE 4
Apparent Kₘ and Vₘₐₓ of ZMO0976 for benzaldehyde,
furfural and xylose in presence of 0.35 mM NADPH

| | Benzaldehyde | Furfural | Xylose |
|---|---|---|---|
| Kₘ (mM) | 1.77 ± 0.11 | 4.15 ± 0.18 | 258 ± 43 |
| Vₘₐₓ (mU/mg protein) | 7200 ± 200 | 5000 ± 1300 | 6900 ± 1200 |

ZMO0976 could also use NADH as cofactor. However, there was marked decrease in its activity as compared to that with NADPH, as shown in Table 5 below.

TABLE 5
Fold reduction in ZMO0976 activity with NADH as cofactor
compared to NADPH as cofactor. 0.35 mM of either
cofactors were used in the assay mixtures.

| | |
|---|---|
| Furfural (10 mM) | 10 |
| Acetaldehyde (260 mM) | 53 |
| Xylose (260 mM) | 18 |

### Discussion of Results

Besides xylose, ZMO0976 readily reduces aromatic aldehydes. In fact, benzaldehyde and furfural are much better substrates than xylose. The affinity to these two aromatic aldehydes is one to two orders of magnitude higher than that of xylose. Intriguingly, neither glucose nor fructose, the two sugars that the Z. mobilis ferments naturally, is a substrate for the enzyme. While not wishing to be bound to any particular theory ZMO0976 may not be allowed to reduce glucose and fructose since this will result in reduction of glucose and fructose available for ED pathway. There has been only one report of a bacterial furfural reductase, but it does not have any activity toward xylose, see, Gutierrez, T., L. O. Ingram, et al. supra. Therefore the enzyme discovered from this work appears to be novel.

As shown in this study, cells with reduced xylose reductase (XR) activities produced less xylitol, suggesting XR is one of the major routes of xylitol synthesis. It is conceivable that xylose metabolism may be benefited by eliminating the XR activity in its entirety. This work, by identifying the XR gene, paves the way for further improvement of xylose fermentation in Z. mobilis through metabolic engineering. For example, by deleting the XR gene, along with GFOR gene, see, Viitanen, McCutchen, et al. supra, xylitol formation could be reduced further and xylose fermentation could be further improved without the impedance of the toxic byproduct. In addition, the increased availability of NADPH cofactor may result in higher biosynthetic activity promoting faster cell growth, see, Miller E., Turner P., et al., “Genetic changes that increase 5-hydroxymethyl furfural resistance in ethanol-producing Escherichia coli LY180”, Biotechnology Letters, 2010, 32: pp. 661-667.

The finding that ZMO0976 reduced furfural and HMF is of significance from a very different perspective. The enzyme potentially provides a detoxification mechanism for cells fermenting lignocellulose for production of ethanol and other products. Furfural and HMF are produced during hydrolysis of lignocellulosic biomass and are potent inhibitors of microbial growth, see, Palmqvist E. & Hahn-Hagerdal B., “Fermentation of lignocellulosic hydrolysates. II: inhibitors and mechanisms of inhibition”, 2000, Bioresource Technology 74: pp. 25-33; and Gutierrez, T., L. O. Ingram, et al. supra. A pre-fermentation step employing recombinant ZMO0976 could be envisioned to reduce both furfural and HMF to concentrations tolerable to a fermenting microorganism in the subsequent process. Alternatively, a microbial strain (not necessarily Z. mobilis) could be engineered to overexpress the ZMO0976, thus endowing cells the ability to better tolerate these two biomass derived inhibitors.

While not wishing to be bound by any theory, it appears that there may be other enzymes in Z. mobilis cells acting as xylose reductase based on the observation that adapted strain A3 (see FIG. 2) harboring mZMO0976 still produces xylitol. One such xylose reductase is glucose-fructose oxidoreductase (GFOR) according to Viitanen, McCutchen, et al. supra.

The nucleotide sequence of mZMO0976 was deposited in GenBank and has an accession number of HQ247815.

The claimed subject matter is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety to the extent that they are not inconsistent and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for treating a lignocellulosic hydrolysate comprising one or more inhibitors, the method comprising: contacting a lignocellulosic hydrolysate comprising one or more inhibitors with an aldo/keto reductase under conditions such that the total amount of inhibitors is reduced.

2. The method of claim 1 wherein the aldo/keto reductase is employed neat, employed in a solution, or generated in situ from one or more cells capable of producing said aldo/keto reductase.

3. The method of claim 2 wherein the aldose/keto reductase is generated in situ from one or more recombinant cells made capable of producing said aldo/keto reductase.

4. The method of claim 1 wherein the aldo/keto reductase comprises the amino acid sequence ZM00976 or a suitable derivative thereof.

5. The method of claim 2 wherein the aldo/keto reductase is generated in situ from one or more recombinant cells made capable of producing said aldose/keto reductase, and wherein the one or more recombinant cells comprise a nucleic acid molecule capable of encoding the amino acid sequence ZM00976 or a suitable derivative thereof.

6. The method of claim 1 wherein the aldo/keto reductase is derived from Zymomonas mobilis.

7. The method of claim 1 wherein the aldo/keto reductase reduces the amount of one or more inhibitors selected from the group consisting of furfural, benzaldehyde, acetaldehyde, and HMF.

8. The method of claim 1 wherein the aldo/keto reductase reduces the amount of two or more inhibitors selected from the group consisting of furfural, benzaldehyde, acetaldehyde, and HMF.

9. The method of claim 1 wherein the aldo/keto reductase reduces the amount of three or more inhibitors selected from the group consisting of furfural, benzaldehyde, acetaldehyde, and HMF.

10. The method of claim 1 wherein the aldose/keto reductase reduces the amount of furfural by converting furfural to 2-furanmethanol.

11. The method of claim 1 wherein one or more cofactors are employed with the aldo/keto reductase.

12. The method of claim 11 wherein the cofactor is NADPH.

13. The method of claim 1 which further comprises fermenting at least a portion of the hydrolysate to produce a bioalcohol.

14. A method for fermenting xylose-containing lignocellulosic hydrolysate, the method comprising: fermenting a xylose-containing lignocellulosic hydrolysate in the presence of
(1) recombinant Z. mobilis which synthesizes mZMO0976 or a suitable derivative thereof in the substantial absence of ZMO0976; or
(2) recombinant Z. mobilis rendered incapable of synthesizing ZMO0976 or a suitable derivative thereof; or
(3) a combination of (1) and (2).

15. An aldo/keto reductase from a recombinant cell wherein the aldo/keto reductase comprises the amino acid sequence ZM00976 and comprises at least one of the following characteristics: (1) a xylose reductase activity of 3400±200 mU/mg protein; (2) a furfural reductase activity of 5470±60 mU/mg protein; (3) a benzaldehyde reductase activity of 4030±250 mU/mg protein; (4) an acetaldehyde reductase activity of 2500±400 mU/mg protein; and (5) ability to reduce HMF present in lignocellulosic biomass.

16. The aldo/keto reductase of claim 15 wherein the aldo/keto reductase comprises at least two of the following characteristics: (1) a xylose reductase activity of 3400±200 mU/mg protein; (2) a furfural reductase activity of 5470±60 mU/mg protein; (3) a benzaldehyde reductase activity of 4030±250 mU/mg protein; (4) an acetaldehyde reductase activity of 2500±400 mU/mg protein; and (5) ability to reduce HMF present in lignocellulosic biomass.

17. The aldo/keto reductase of claim 15 wherein the aldo/keto reductase comprises at least three of the following characteristics: (1) a xylose reductase activity of 3400±200 mU/mg protein; (2) a furfural reductase activity of 5470±60 mU/mg protein; (3) a benzaldehyde reductase activity of 4030±250 mU/mg protein; (4) an acetaldehyde reductase activity of 2500±400 mU/mg protein; and (5) ability to reduce HMF present in lignocellulosic biomass.

18. A recombinant cell capable of producing an aldo/keto reductase useful for treating a lignocellulosic hydrolysate wherein the recombinant cell comprises a nucleic acid molecule capable of encoding the amino acid sequence ZM00976 or a suitable derivative thereof.


SEQUENCE LISTING (2 sequences)

SEQ ID NO: 1 - protein, 340 aa, Artificial Sequence
  sequence of aldo/keto reductase ZMO0976 (see Fig. 4A)
MNTSTQKPAHFDKISIKGIDKSATRVALGTWAIGGWMWGGTDDDASIKTIHRAIDLGINIIDTAPAYGRGHAEEVVGKAIKGQRDNLIIATKVGLDWTLTPDQSMRRNSSASRIKKEIEDSLRRLGTDYIDLYQVHWPDPLVPIEETATILEALRKEGKIRSIGVSNYSVQQMDEFKKYAELAVSQSPYNLFEREIDKDILPYAKKNDLVVLGYGALCRGLLSGRMTADRAFTGDDLRKTDPKFQKPRFEHYLAAVEELKKLAKEHYNKSVLALAIRWMLEQGPTLALWGARKPEQIDGIDEVFGWQISDEDLKQIDAILAKNIPNPIGAEFMAPPPRDK

SEQ ID NO: 2 - protein, 340 aa, Artificial Sequence
  sequence of aldo/keto reductase mZMO0976 (see Fig. 4B)
MNTSTQKPAHFDKISIKGIDKSATRVALGTWAIGGWMWGGTDDDASIKTIHRAIDLGINIIDTAPAYGRGHAEEVVGKAIKGQRDNLIIATKVGLDWTLTPDQSMRRNSSASRIKKEIEDSLRRLGTDYIDLYQVHWPDPLVPIEETATILEALRKEGKIRSIGVSNYSVQQMDEFKKYAELAVSQSPYNLFEREIDKDILPYAKKNDLVVLGYGALCRGLLSGRMTADRAFTGDDLRKTDPKFQKPRFEHYLAAVEELKKLAKEHYNKSVLALAIRWMLEQGPTLALWGACKPEQIDGIDEVFGWQISDEDLKQIDAILAKNIPNPIGAEFMAPPPRDK